(19) United States
(12) Patent Application Publication
(10) Pub. No.: US 2026/0065552 A1
(43) Pub. Date: Mar. 5, 2026

(54) RECOVERY-BASED BLACK BOX DETECTION OF AI-GENERATED CONTENT FOR MEDICAL DECISION MAKING

(71) Applicant: NEC Laboratories America, Inc., Princeton, NJ (US)

(72) Inventors: Yiyou Sun, Madison, WI (US); Wei Cheng, Princeton Junction, NJ (US); Haifeng Chen, West Windsor, NJ (US)

(21) Appl. No.: 19/303,677

(22) Filed: Aug. 19, 2025

Related U.S. Application Data
(60) Provisional application No. 63/687,421, filed on Aug. 27, 2024.

(51) Int. Cl. G06T 11/60; G06T 7/00; G16H 50/20

(52) U.S. Cl. CPC G06T 11/60; G06T 7/0014; G16H 50/20; G06T 2210/41

(57) ABSTRACT

Methods and systems include fine-tuning a surrogate model, using example images generated by a target model, to match a distribution of the target model. A new image is masked to generate a masked image. A recovered image is generated that fills in a masked region of the masked image using the surrogate model. The recovered image is compared to the new image to determine that the new image was generated by the target model. An action is performed responsive to the determination that the new image was generated by the target model.

### RELATED APPLICATION INFORMATION

This application claims priority to U.S. Patent Application No. 63/687,421, filed on Aug. 27, 2024, incorporated herein by reference in its entirety.

### BACKGROUND

### Technical Field

The present invention relates to detecting artificial intelligence (AI)-generated content and, more particularly, to black box detection of such content.

### Description of the Related Art

The rapid advancement of generative AI models has led to significant progress in synthesizing photorealistic images. This technology raises concerns about potential misuse, such as the creation of convincing fake news. Existing methods of detecting AI-generated content often struggle with practical implementation due to a need for extensive datasets of both real and fake images or for information about the generative model's internals.

### SUMMARY

A method includes fine-tuning a surrogate model, using example images generated by a target model, to match a distribution of the target model. A new image is masked to generate a masked image. A recovered image is generated that fills in a masked region of the masked image using the surrogate model. The recovered image is compared to the new image to determine that the new image was generated by the target model. An action is performed responsive to the determination that the new image was generated by the target model.

A system includes a hardware processor and a memory that stores a computer program. When executed by the hardware processor, the computer program causes the hardware processor to fine-tune a surrogate model, using example images generated by a target model, to match a distribution of the target model, to mask a new image to generate a masked image, to generate a recovered image that fills in a masked region of the masked image using the surrogate model, to compare the recovered image to the new image to determine that the new image was generated by the target model, and to perform an action responsive to the determination that the new image was generated by the target model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a system for determining whether an input image was generated by an artificial intelligence (AI) model, in accordance with an embodiment of the present invention;

FIG. 2 is a block/flow diagram of a method for determining whether an input image was generated by an AI model, in accordance with an embodiment of the present invention;

FIG. 3 is a block/flow diagram of a method for identifying and responding to AI-generated content, in accordance with an embodiment of the present invention;

FIG. 4 is a block diagram of a healthcare facility where AI-generated content detection is used to assist with medical decision making, in accordance with an embodiment of the present invention;

FIG. 5 is a block diagram of a computing device that can be used to detect AI-generated content, in accordance with an embodiment of the present invention;

FIG. 6 is a diagram of an exemplary neural network architecture that can be used to implement part of a surrogate model, in accordance with an embodiment of the present invention; and

FIG. 7 is a diagram of an exemplary deep neural network architecture that can be used to implement part of a surrogate model, in accordance with an embodiment of the present invention.

### DETAILED DESCRIPTION OF PREFERRED EMBODIMENTS

Black box detection of artificial intelligence (AI)-generated images identifies such content even when access to the generating model's internals is not available. In black box scenarios, only inputs to, and outputs from, the generative model are available. A combination of a distribution-aligned surrogate model and inpainting analysis may be used. The surrogate model is fine-tuned, using a dataset of images generated by the target model, to mimic the target model's distribution. Inpainting analysis identifies discrepancies in masked and reconstructed image regions, allowing for accurate classification of images as being real or being AI-generated.

This approach is adaptable to new generative models, making it possible to quickly align the surrogate model's distribution with that of the target model to ensure consistent performance. It is furthermore scalable, as it needs only a small amount of data and computational resources, which makes it practical for widespread application across various industries including social media, journalism, and forensic analysis.

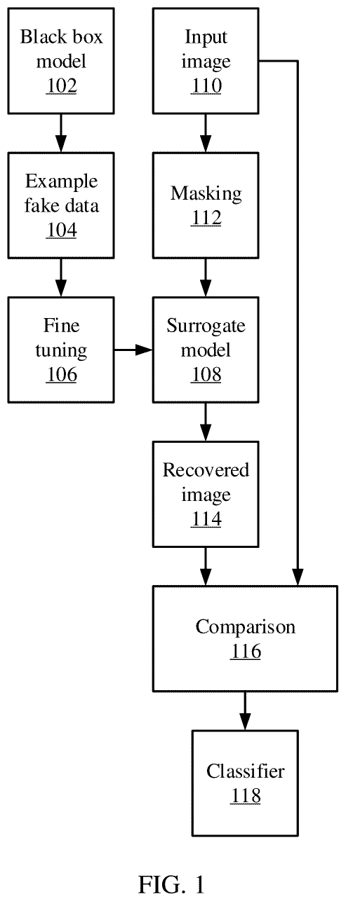

Referring now to FIG. 1, a system for identifying AI-generated content is shown. A surrogate model 108 is trained to mimic the behavior of a black box model 102. This process includes generating a set of example fake data 104 using the black box model 102 and fine-tuning 106 the surrogate model 108. The fine-tuning 106 seeks to train the surrogate model 108 to replicate the statistical distribution of the black box model 102, such that its outputs will share characteristics with the example fake data 104.

During operation, an input image 110 is masked 112 to create a masked image. The masked image is input to the surrogate model 108 with instructions to fill in the masked region, a task referred to as inpainting. This generates a recovered image 114, which is compared 116 to the original input image 110. If the input image 110 was created by the black box model 102, then the recovered image 114 will tend to be similar to the input image 110. If they differ significantly, then it can be concluded that the input image 110 was not generated by the black box model 102. A classifier 118 then makes a determination whether the original image was generated by the black box model 102 or not.

The task can thus be framed as a binary classification problem. Only a limited amount of information about the black box model 102 is assumed. For example, a diffusion probabilistic model is a parameterized Markov chain that is trained using variational inference to produce samples matching the training data after a finite time. This process gradually diffuses a sample from the distribution and learns to reverse the diffusion process.

In the diffusion (or forward) process for denoising diffusion probabilistic models (DDPMs), a sample x₀ may be an image. The sample is repeatedly corrupted using Gaussian noise in sequential steps t=1, . . . , T according to a monotonically increasing noise schedule

$${\{ β \}}_{t = 1}^{T} :$$

$$q ( {x}_{t} \vert {x}_{0} ) = 𝒩 ( \sqrt{1 - {β}_{t}} {x}_{t - 1} , {β}_{t} I )$$

With αₜ=1−βₜ and

$${\overline{α}}_{t} = {\prod }_{s = 1}^{t} {α}_{s}$$

a sample can be drawn directly from the forward process at arbitrary times:

$$q ( {x}_{t} \vert {x}_{0} ) = 𝒩 ( \sqrt{{\overline{α}}_{t}} {x}_{0} , {( 1 - {\overline{α}}_{t} )}_{t} I )$$

The noise schedule may be designed to satisfy q(xₜ|x₀)≈ [symbol] (0, I). During a denoising (or reverse) process, samples may be drawn iteratively from q(xₜ₋₁|xₜ) to obtain a clean image from x_{T}˜ [symbol] (0, I). However, q(xₜ₋₁|xₜ) is intractable, as it depends on the entire underlying data distribution, and so it may be approximated by a deep neural network. More formally, q(xₜ₋₁|xₜ) may be approximated by:

$${p}_{θ} ( {x}_{t - 1} \vert {x}_{t} ) = 𝒩 ( {μ}_{θ} ( {x}_{t} , t ) , {\sum }_{θ} ( {x}_{t} , t ) )$$

where mean μ_{θ} and covariance Σ_{θ} are given by the output of the model, or the latter may be set to a constant.

The surrogate model 108 may be expressed as [symbol] and the target black box model 102 may be expressed as [symbol] . Images that are masked after being generated by the black box model are more likely to be recovered accurately by a distribution-aligned surrogate model. In contrast, real images are more difficult to reconstruct effectively due to their intrinsically complex characteristics. There is a significant difference in the recovery quality of real images as compared to those generated by the black box model 102.

Given an input image x, the goal is to determine whether it is synthesized by a generative model or if it is a real image. A mask m divides the image into two parts: the known pixels (1−m)⊙x and the unknown pixels m⊙x. The distribution-aligned surrogate model 108 is applied to recover the unknown pixels m⊙x′ conditioned on the known pixels. The difference between m⊙x and m⊙x′ helps distinguish between real and generated images. A metric δ may be computed for this discrepancy gap and may be used as a scoring function to classify the input image 110 as being either real or generated, for example by comparing the score to a threshold value. The scoring function may be implemented using any appropriate metric, such as the L1 distance, the L2 distance, the structural similarity index measure, or the peak signal-to-noise ratio.

In some embodiments, the recovery results may be sampled multiple times to account for the stochastic nature of the process and to obtain a more robust evaluation. Thus multiple recovered images may be generated with different randomly influenced outcomes. In such embodiments, an average of the score of the discrepancy gap of the samples may be used as the final score for the input image 102.

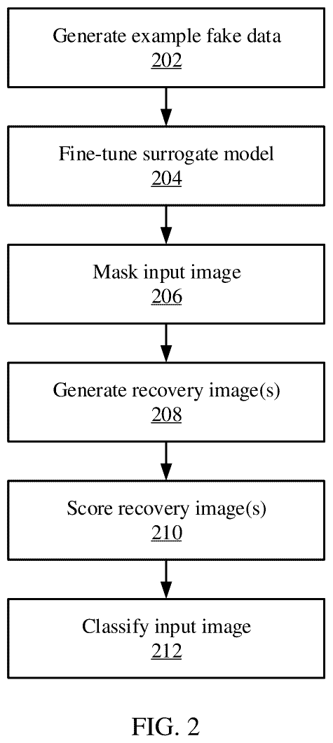

Referring now to FIG. 2, a method of identifying AI-generated images is shown. Block 202 generates example fake data 104 using the black box model 102. Any appropriate number of fake data images may be generated in this fashion, for example prompting the black box model 102 to generate photorealistic images in a variety of different scenarios.

Block 204 then uses the example fake data 104 to fine-tune the surrogate model 108. The surrogate model 108 is initially selected as a best-guess of how the black box model 102 operates and may be pre-trained using a general purpose dataset.

Block 206 masks an input image, which is being tested. A subset of the pixels in the input image are masked, for example by replacing them with black pixels. The mask may include a continuous set of pixels in the input image or may include discrete sets of pixels. In some cases, block 206 may generate multiple different masked versions of the input image 102, for example by masking different regions of the input image. The mask may be selected randomly. In some embodiments, the mask may include a single rectangle. In some embodiments the mask may include multiple randomly generated shapes. In some embodiments the mask may block alternating lines or any other appropriate pattern.

Block 208 uses the fine-tuned surrogate model 108 to attempt to recover the input image 102 from the masked image(s). In some cases multiple recovery images may be generated for multiple respective masked images. In some cases block 208 may generate multiple recovery images for each masked image. Block 210 scores the recovery image(s) according to the metric δ and block 212 classifies the input image using the scores.

There will be a distribution gap between the surrogate model 108 and the black box model 102. The goal of fine-tuning in block 204 is to bring the distribution of the surrogate model 108 as close as possible to that of the black box model 102. To align the distribution of [symbol] with that of [symbol] , a dataset

$$S = {\{ {x}_{i} \}}_{i = 1}^{N}$$

of N images is generated by the black hole model 102. This alignment dataset is used for fine-tuning to train the surrogate model 108 to create images similar to those created by the black box model 102.

A low rank adaptation may be used for the surrogate model to enable parameter-efficient fine-tuning. The model [symbol] +θ is trained with the dataset S, while the pretrained parameters of the original surrogate model 108 are kept fixed. After training, the aligned model [symbol] +θ generates a distribution similar to that of the target black hole model [symbol] .

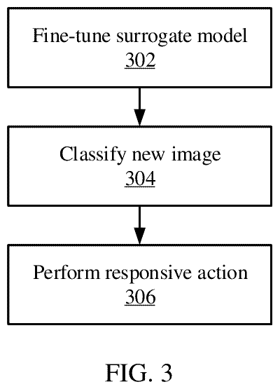

Referring now to FIG. 3, a method of detecting and responding to AI-generated content is shown. Block 302 fine-tunes the surrogate model 108 as described above, based on example fake data 104 generated by a black box model 102. Block 304 then uses the fine-tuned surrogate model 108 to classify a new image. This produces a determination of whether the new image was generated by the black box model 102.

In some cases the fine-tuning 302 and the classification 304 are performed by the same entity, but they may instead be performed by different entities. For example, the surrogate model 108 may be fine-tuned by a vendor and may then be deployed to one or more different locations, so that the image classification may be performed locally by a customer.

When the new image is classified as being generated by the black box model 102, block 306 performs a responsive action. This responsive action may depend on the context and purpose of the image. In some cases the new image may be deleted or may be flagged as including misinformation or fake news. In some cases, where the new image is received in a medical context and indicates an injury or symptom, the new image may be flagged or discarded as misrepresenting the health condition of a patient.

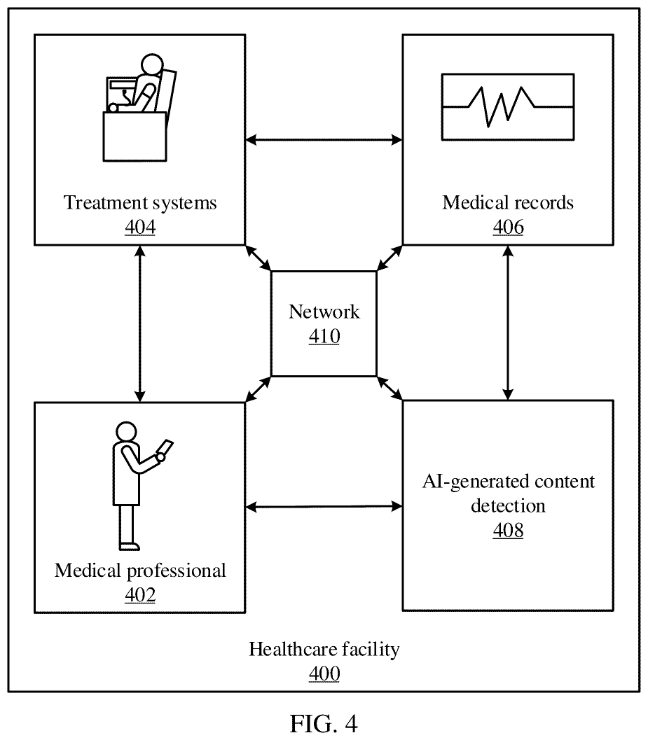

Referring now to FIG. 4, a diagram of image analysis with region splitting is shown in the context of a healthcare facility 400. AI-generated content detection 408 may be used to review information supplied by patients and other sources to verify that they accurately represent the patient's condition. For example, images taken by the patient may be processed to determine whether they are generated by an AI model, to determine whether these images can be used for the purposes of diagnosis.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Based on information drawn from the AI-generated content detection 408, the medical professionals 402 may then make medical decisions about patient healthcare suited to the patient's needs. For example, the medical professionals 402 may make treatment decisions based on images relating to the patient's condition after determining that they were not generated by an AI model and may prescribe particular medications, surgeries, and/or therapies that are appropriate to the diagnosis disease.

The different elements of the healthcare facility 400 may communicate with one another via a network 410, for example using any appropriate wired or wireless communications protocol and medium. Thus AIO-generated content detection 408 receives data from treatment systems 404, medical professionals 402, and from medical records 406, and analyzes images in the medical records 406 to determine whether they are generated by an AI model. The AI-generated content detection 408 may further coordinate with treatment systems 404 in some cases to automatically administer or alter a treatment. For example, if the image analysis indicates a particular image was generated by an AI model, this may indicate that a diagnosis relying on the image was incorrect, and the system may automatically change the treatment, such as by initiating or halting the administration of a medication.

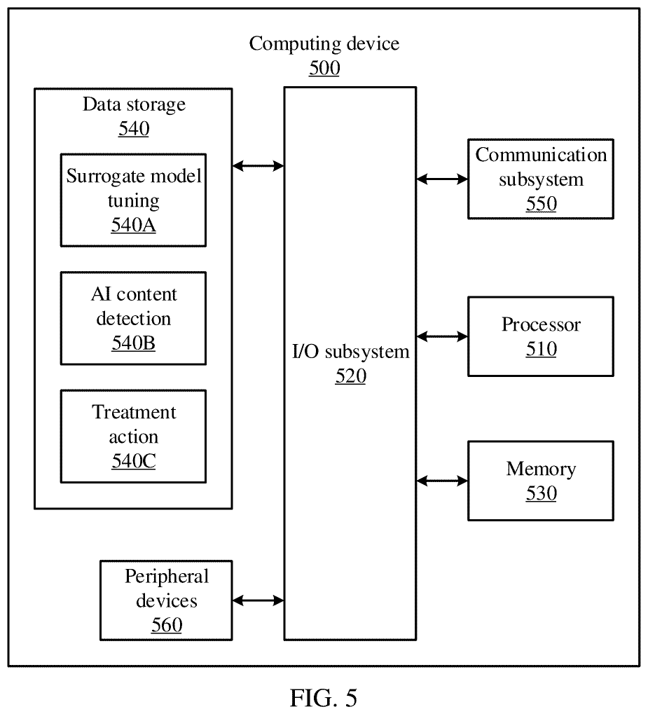

Referring now to FIG. 5, an exemplary computing device 500 is shown, in accordance with an embodiment of the present invention. The computing device 500 is configured to perform visual question answering.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for fine-tuning the surrogate model, 540B for detecting AI-generated content, and/or 540C for performing treatment actions. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

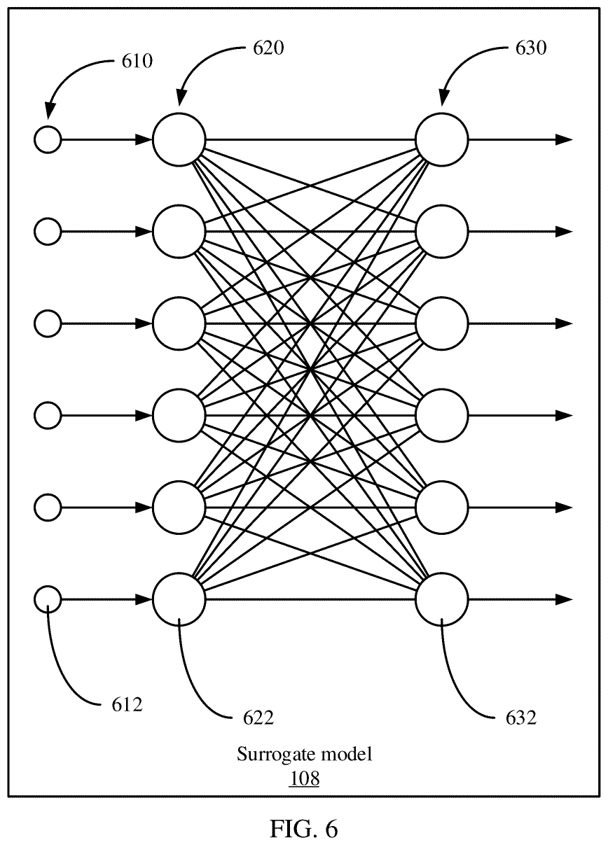

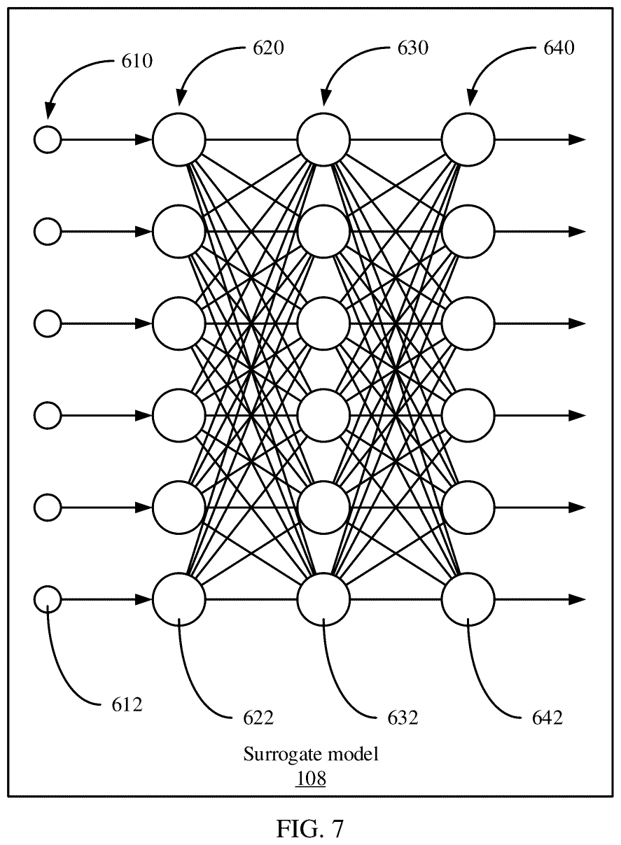

Referring now to FIGS. 6 and 7, exemplary neural network architectures are shown, which may be used to implement parts of the present machine learning models, such as the surrogate model 108. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
fine-tuning a surrogate model, using example images generated by a target model, to match a distribution of the target model;
masking a new image to generate a masked image;
generating a recovered image that fills in a masked region of the masked image using the surrogate model;
comparing the recovered image to the new image to determine that the new image was generated by the target model; and
performing an action responsive to the determination that the new image was generated by the target model.

2. The method of claim 1, wherein masking includes generating a plurality of masked images with differing masked regions.

3. The method of claim 2, wherein generating the recovered image includes generating a plurality of recovered images for respective masked images of the plurality of masked images.

4. The method of claim 1, wherein generating the recovered image includes generating a plurality of different recovered images that fill in the masked region of the masked image using the surrogate model.

5. The method of claim 4, wherein comparing the recovered image to the new image includes generating a score for each of the plurality of different recovered images and averaging the scores.

6. The method of claim 1, wherein masking the new image includes masking discrete sets of pixels from the new image.

7. The method of claim 1, wherein the surrogate model is a pretrained diffusion machine learning model and wherein fine-tuning the surrogate model includes low rank adaptation that keeps parameters of the pretrained diffusion model fixed.

8. The method of claim 1, wherein the new image indicates a health condition of a patient.

9. The method of claim 8, wherein the action includes flagging the new image as being generated by artificial intelligence to assist with medical decision making.

10. The method of claim 8, wherein the action includes performing a treatment action for the patient.

11. A system, comprising:
a hardware processor; and
a memory that stores a computer program which, when executed by the hardware processor, causes the hardware processor to:
fine-tune a surrogate model, using example images generated by a target model, to match a distribution of the target model;
mask a new image to generate a masked image;
generate a recovered image that fills in a masked region of the masked image using the surrogate model;
compare the recovered image to the new image to determine that the new image was generated by the target model; and
perform an action responsive to the determination that the new image was generated by the target model.

12. The system of claim 11, wherein the masking includes generation of a plurality of masked images with differing masked regions.

13. The system of claim 12, wherein generation of the recovered image includes generation of a plurality of recovered images for respective masked images of the plurality of masked images.

14. The system of claim 11, wherein generation of the recovered image includes generating a plurality of different recovered images that fill in the masked region of the masked image using the surrogate model.

15. The system of claim 14, wherein comparison of the recovered image to the new image includes generation of a score for each of the plurality of different recovered images and averaging the scores.

16. The system of claim 11, wherein the masking of the new image includes masking discrete sets of pixels from the new image.

17. The system of claim 11, wherein the surrogate model is a pretrained diffusion machine learning model and wherein the fine-tuning of the surrogate model includes low rank adaptation that keeps parameters of the pretrained diffusion model fixed.

18. The system of claim 11, wherein the new image indicates a health condition of a patient.

19. The system of claim 18, wherein the action includes flagging the new image as being generated by artificial intelligence to assist with medical decision making.

20. The system of claim 18, wherein the action includes a treatment action for the patient.
